(19) United States
(12) Patent Application Publication
(10) Pub. No.: US 2016/0184024 A1
(43) Pub. Date: Jun. 30, 2016

(54) CleanSteth Disposable Stethoscope Cover

(71) Applicant: Dephillia McClenon, Houston, TX (US)

(72) Inventor: Dephillia McClenon, Houston, TX (US)

(21) Appl. No.: 14/585,313

(22) Filed: Dec. 30, 2014

(51) Int. Cl. A61B 19/02

(52) U.S. Cl. CPC A61B 19/026

(57) ABSTRACT

A stethoscope covers for protecting the patient from cross contamination between users. The cover includes a flexible thin plastic ring with a one inch opening, a top edge of 0.25″ with adhesive for the non-woven material to be attached.

### FIELD OF THE INVENTION

The present invention relates generally to head coverings and in particular, to the stethoscopes.

### BACKGROUND OF THE INVENTION

On a daily basis the medical profession uses their stethoscope to check patient's heart, blood pressure lungs and other body vital signs. Most health care professions are aware of the need to have a clean stethoscope head with diagnosing patients; to reduce or eliminate cross contamination between patients. There is no real solution to the problem without affecting the sound of the diaphragm. A need, therefore, exists for a cover that may be applied to the stethoscope that provides protection without impeding the sound quality of the stethoscope.

In light of the above mentioned problem, it is the principal object of the present invention to provide a cover for the stethoscope, which can prevent cross contamination between patients and provide clear concise sound.

It is an additional object of the invention to provide a stethoscope of the type described which may be positioned upon the stethoscope in a comfortable and secure manner without impeding the sound of the diaphragm.

It is an object of the invention to provide improved elements and arrangements of the stethoscope cover thereof in an stethoscope cover for the purpose described which is lightweight in construction, inexpensive in manufacture, and fully effective in use.

Briefly, the stethoscope cover in accordance with this invention achieves the intended objects by featuring a flexible sheet of moisture resistant non-woven material adhered by a glue like substance to a flexible thin plastic ring.

The foregoing and other objects, features and advantages of the present invention will become readily apparent upon further review of the following detailed description of the preferred embodiment as illustrated in the accompanying drawings.

### BRIEF DESCRIPTION OF THE DRAWINGS

The present invention may be more readily described with the reference to the accompanying drawings, in which:

FIG. 1 is a perspective view of a stethoscope cover in accordance with the present invention shown positioned upon the stethoscope diaphragm.

FIG. 2 is a side elevation view of the stethoscope cover having portions broken away to reveal the interior details thereof.

FIG. 3 is a front elevation view of the stethoscope cover.

Similar reference characters denote corresponding features consistently throughout the accompanying drawings.

### DETAILED DESCRIPTION OF THE PREFERRED EMBODIMENT

Referring now to the Figs., a stethoscope cover in accordance with the present invention is shown at . . . The stethoscope cover . . . includes a thin plastic flexible ring of different diameters with a top of 0.25″ with an applied adhesive to adhere the non-woven moisture resistant material to the top of the ring.

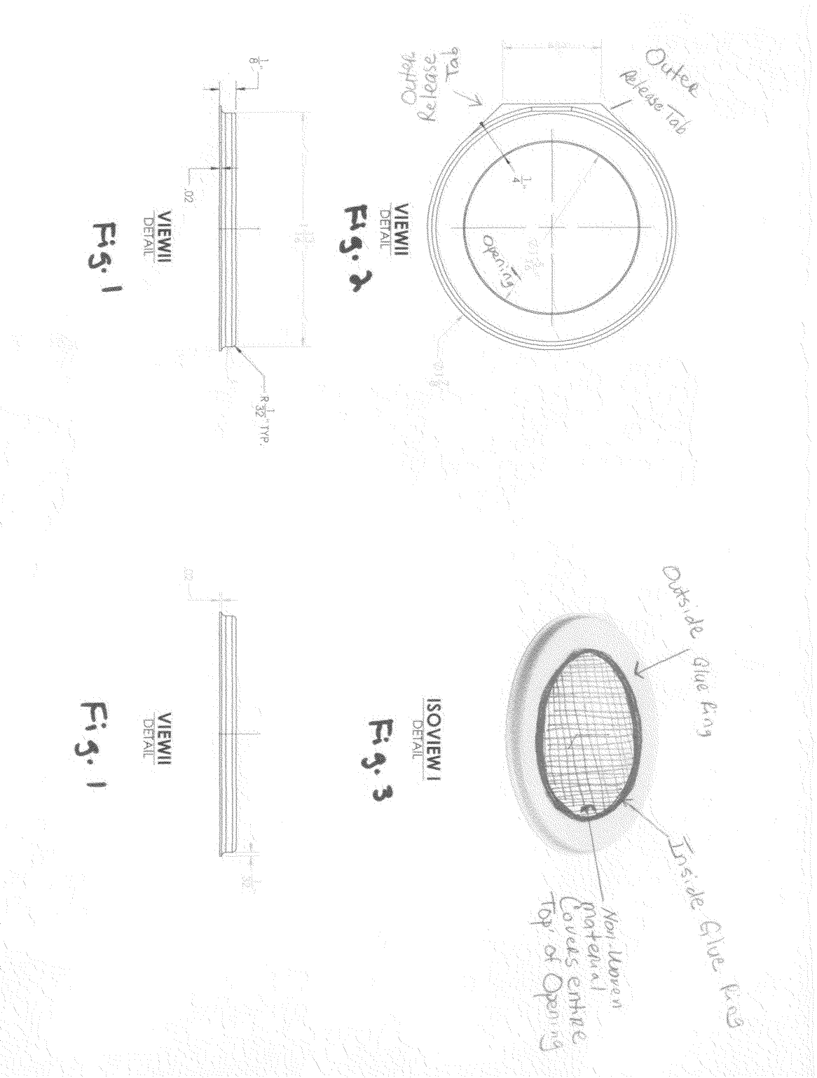

The finished stethoscope . . . may be easily positioned upon the diaphragm of the stethoscope . . . as shown in FIG. 1.

The non-woven moisture resistant material prevents any cross contamination from being transferred to other patients. After use, the cover should be removed from the diaphragm of the stethoscope and properly discarded.

While the inventive cover has been described with a degree of particularity, it will be appreciated by those skilled in the art of modifications may be made thereof. For example, the thin plastic ring can be made to fit different sizes of stethoscope diaphragms. Further, the thin plastic ring may be tailored to a more precise fit depending on the brand of stethoscope.

Therefore, it is to be understood that the present invention is not limited to the sole embodiment described above, but encompasses any and all embodiments within the scope of the following claims.

I claim:

1. A cover for the diaphragm, compromising: a flexible thin plastic ring having an opening top of one inch in diameter with an edge 0.25″ and adhesive over the top 0.25″ opening of the ring, which adheres the nonwoven disc to fit over the opening of the ring.

2. The stethoscope covers according to claim 1 wherein the nonwoven disc is said to be adhered to the top opening of one inch in diameter an adhesive the ring attached to the 0.25″ edge, adjoin the non-woven material to the top of the thin flexible plastic ring.

3. The stethoscope covers according to claim 2 where in the top layer of non-woven material will be secured by adhesive to the flexible thin plastic ring.
